(19) United States
(12) Patent Application Publication
(10) Pub. No.: US 2009/0240099 A1
(43) Pub. Date: Sep. 24, 2009

(54) BI-MODAL COCHLEA STIMULATION

(75) Inventor: Brian M. Conn, Broomfield, CO (US)

Correspondence Address: MARSH, FISCHMANN & BREYFOGLE LLP, 8055 East Tufts Avenue, Suite 450, Denver, CO, 80237, US

(73) Assignee: Otologics, LLC, Boulder, CO (US)

(21) Appl. No.: 12/395,307

(22) Filed: Feb. 27, 2009

Related U.S. Application Data
(60) Provisional application No. 61/032,812, filed on Feb. 29, 2008.

(51) Int. Cl. H04R 25/00; A61F 11/04; A61N 1/36

(52) U.S. Cl. 600/25; 607/57

(57) ABSTRACT

An improved implantable hearing instrument and associated method utilize a transducer to mechanically stimulate a patient's cochlea (e.g. via the round window or oval window) in response to a first electrical drive signal, and a supply electrode to electrically stimulate the patient's cochlea in response to a second drive signal. The first and second drive signals may be provided to affect mechanical stimulation across a first predetermined frequency range and electrical stimulation across a second predetermined frequency range, respectively, wherein the predetermined frequency ranges are at least partially non-overlapping. In one embodiment an electromechanical transducer, having the supply member supportably interconnect thereto, may be selectively positioned via a mounting member fixedly interconnected to a patient's skull. The supply electrode may define a distal tip that is supportably interconnected to a vibratory member of the electromechanical transducer. In another embodiment, an implantable transducer may be employed that includes an external housing and an active transducer element located within an internal chamber of the external housing for receiving the first electrical signal. Further, the supply electrode may be one of electrically connected to and defined by at least an electrically-conductive portion of the external housing of the transducer.

### RELATED APPLICATIONS

This application claims priority to U.S. Provisional Application Ser. No. 61/032,812, filed on Feb. 29, 2008, entitled “BI-MODAL COCHLEA STIMULATION”, the entirety of which is hereby incorporated by reference.

### FIELD OF THE INVENTION

The present invention relates to implantable hearing instruments, and more particularly, to a bi-modal, implantable hearing instrument adapted for mechanical and electrical stimulation of the cochlea.

### BACKGROUND OF THE INVENTION

The utilization of implanted hearing instruments is ever-increasing. In this regard, implantable hearing instruments provide operative and cosmetic advantages relative to conventional ear canal hearing instruments.

Typically, an implanted hearing instrument may comprise implanted componentry for mechanically stimulating a middle ear component of a patient's auditory system, or alternatively, for electrically stimulating an inner ear component of a patient's auditory system. As may be appreciated, depending on patient-specific needs, both approaches have relative advantages and disadvantages. Further, in either approach the implantation of componentry entails a surgical procedure with attendant surgical personnel and facility requirements.

To facilitate increased utilization of implanted hearing instruments, the present inventor has recognized the desirability of providing an approach which realizes the benefits of both mechanical stimulation and electrical stimulation of a patient's auditory system, and which also facilitates the efficient and reliable surgical positioning of implantable hearing instrument componentry.

### SUMMARY OF THE INVENTION

An implantable hearing instrument comprising the present invention may include a transducer for providing a vibratory output (e.g. in response to a first electrical drive signal corresponding with an acoustic signal), and a supply electrode for providing an electrical output (e.g. in response to a second electrical drive signal corresponding with the acoustic signal). The vibratory output of the transducer is employable for direct mechanical stimulation of a patient's cochlea, and the electrical output of the supply electrode is employable for direct electrical stimulation of a patient's cochlea, wherein enhanced bi-modal cochlea stimulation may be realized.

For example, enhanced perception of acoustic signals, or hearing assistance, may be achieved over a relatively wide acoustic frequency range. Additionally, or alternatively, enhanced bi-modal stimulation may be realized to lessen patient discomfort associated with tinnitus via enhanced “masking”. In this regard, the apparatus and methods of the present invention are employable to realize enhanced hearing assistance and/or tinnitus treatment.

In one aspect, the transducer may be an electromechanical transducer, and the supply electrode may be supportably interconnected to or otherwise in vibratory engagement with the electromechanical transducer. In this regard, the electromechanical transducer may comprise a housing and a vibratory member that is supportably interconnected to the housing for movement relative thereto to communicate the vibratory output. In turn, the supply electrode may be supportably interconnected to, defined by or in vibratory engagement with the vibratory member.

In one arrangement, the supply electrode may define a distal end for engaging a patient's cochlea, wherein both electrical stimulation and mechanical stimulation of a patient's cochlea are realized via the distal end. In one approach, the distal end may comprise an electrically-conductive material and may include an arcuate surface for engaging an outer surface of a patient's cochlea. By way of example, a bulbous surface may be sized and positioned to engage a round window membrane, oval window membrane, a bony exterior, a semicircular canal wall or artificial fenestration of a patient's cochlea.

In another approach, the distal end may be adapted for partial insertion into a patient's cochlea. For example, the distal end may be reduced in cross-section (e.g. tapered down) to facilitate penetration/insertion and advancement through a small surgical incision on a patient's cochlear component, e.g. an oval window membrane or round window membrane (e.g. wherein the supply electrode extends from outside to inside the cochlea). In this approach, a small amount of fascia or other autologous tissue may be disposed around the supply electrode to sealably interconnect the supply electrode to the surrounding cochlear tissue after surgical placement. Further, in this approach the electrode may take the form of a prosthetic piston and a proximal end of the supply electrode may include a bail for selective interconnection to a vibratory member either prior to or after surgical placement of the supply electrode.

In another arrangement, a supply electrode may include a distal end and a proximal portion supportably interconnected or having a bail for selective interconnection to a vibratory member. The proximal portion of the vibratory member may also be adapted (e.g. reduced in cross-section) for partial insertion into a patient's cochlea. For example, the proximal portion may be tapered down to facilitate penetration/insertion and advancement through a small incision on a patient's cochlear component, e.g. an oval window membrane or round window membrane (e.g. wherein the supply electrode extends from outside to inside the cochlea). Again, a small amount of fascia or other autologous tissue may be disposed around the supply electrode to sealably interconnect the supply electrode to the surrounding oval window or round window tissue after surgical placement. The distal portion of the supply electrode may be flexible, jointed and/or otherwise curved for inserted positioning within a curved portion of a patient's cochlea. Further, the distal portion of the supply electrode may comprise a plurality of electrode elements spaced along the distal portion. In this regard, the drive signal supplied to the supply electrode may be provided to drive the plurality of electrode elements to affect electrical stimulation across a corresponding plurality of different frequency ranges.

In another aspect, an implantable hearing instrument may comprise a mounting member that is interconnectable in fixed relation to a patient's skull, and that is otherwise adapted for supportable interconnection of an electromechanical transducer thereto. Further, the instrument may include a positioning means, interconnectable between the electromechanical transducer and mounting member, for selectively locating the electromechanical transducer, vibratory member and supply electrode in a desired fixed position relative to a patient's cochlea.

In another aspect, the implantable hearing instrument may be provided so that vibratory output of the transducer mechanically stimulates a patient's cochlea across a first frequency range in response to the first electrical signal, and so that the supply electrode electrically stimulates a patient's cochlea across a second frequency range in response to the second electrical signal. In turn, the first and second frequency ranges may be established to be at least partially non-overlapping. In this regard, at least a portion of the first frequency range may comprise frequencies which are higher than those included in the second frequency range, and at least a portion of the second frequency range may include frequencies which are lower than those induced within the first frequency range. Further, in certain implementations, at least portions of the first and second frequency ranges may be provided to be overlapping.

In another aspect the transducer may include an external housing that defines a hermetically-sealed internal chamber therewithin, and an active transducer element located within the internal chamber for receiving the first electrical drive signal. Further, the supply electrode may be at least one of electrically interconnected to and defined by at least an electrically-conductive portion of the external housing.

In one approach, the transducer may comprise a floating mass transducer. In one implementation, the active element of the floating mass transducer may comprise a coil element fixedly interconnected to the housing. In another implementation, the active transducer element of the floating mass transducer may comprise a piezoelectric element. In both implementations, a mass may be disposed within the housing, wherein upon receipt of the first drive signal the active element moves relative to the mass causing the housing to vibrate. In turn, the housing may be disposed in physical contact with a patient's cochlea to yield mechanical stimulation.

In conjunction with the utilization of a floating mass transducer, the supply electrode may be defined by an electrically conductive portion of the external housing that is disposed to physically contact a patient's cochlea. In turn, the conductive portion of the housing may both electrically and mechanically stimulate a patient's cochlea. In a streamline arrangement, the external housing may be entirely electrically-conductive. In another approach, the transducer may comprise an electromechanical transducer having a vibratory member that is supportably interconnected to the external housing for movement relative thereto in response to the vibratory output. In this regard, the supply electrode may be supportably interconnected to the vibratory member. In one implementation, the supply electrode may define a distal end for engaging a patient's cochlea. For example, the distal end may be adapted for externally contacting the round window or oval window of a patient's cochlea. In another implementation, the distal end may be adapted for insertion through a patient's oval window or round window.

As may be appreciated, the present invention also comprises methods for stimulating a patient's cochlea with an implantable hearing instrument, wherein the methods include the steps of generating a vibratory output at a transducer to mechanically stimulate a patient's cochlea in response to a first electrical signal corresponding with an acoustic signal, and providing an electrical output at a supply electrode to electrically stimulate the patient's cochlea in response to a second electrical signal corresponding with the acoustic signal. The method may further comprise the step of applying the vibratory output directly to a patient's cochlea. Similarly, the providing step may include the further steps of directly engaging a patient's cochlea with a supply electrode, and conveying the second electrical signal to the supply electrode.

In one aspect, the applying step may comprise contacting a patient's cochlea with at least one of a vibratory member operatively interconnected to an electromechanical transducer and a supply electrode supportably carried by such a vibratory member. Such contact may be realized via a number of different approaches.

In one approach, the method may include inserting at least a distal portion of the at least one of the vibratory member and the supply electrode into a predetermined component of the patient's cochlea. In one implementation, the supply electrode may be supportably and distally mounted to a vibratory member, wherein the engaging step entails engagement of at least a portion of the supply electrode inside of the patient's cochlea. In another implementation, the supply electrode may be disposed to extend through and beyond a distal portion of the vibratory member, wherein a distal portion of the supply electrode is one of flexible, jointed and curved for positioning with a curved portion of a patient's cochlea. In yet another implementation, the supply electrode may be integrally defined by a vibratory member.

In another approach, the method may comprise engaging an external surface of a predetermined component of a patent's cochlea with at least one of a vibratory member and supply electrode. In one implementation, the supply electrode may be supportably and distally mounted to the vibratory member. In another approach, the supple electrode may be integrally defined by a vibratory member.

In another aspect, the vibratory output may be generated at a transducer having an external housing and an active transducer element located within an internal chamber of the external housing. In conjunction with this aspect, supply electro may be at least one of electrically connected to and defined by an electrically conductive portion of the external housing of the transducer. In one approach, the supply electrode may be defined by the electrically conductive portion of the external housing, wherein the engaging step includes contacting a patient's cochlea with the electrically conductive portion of the external housing. By way of example, the transducer may comprise a floating mass transducer, wherein the active transducer element is one of a coil element and a piezoelectric element. In turn, the floating mass transducer may include a mass, wherein the applying step includes moving the active transducer element relative to the mass to vibrate the external housing.

In another approach, the supply electrode may be supportably interconnected to and movable relative to an electromechanical transducer. In turn, the vibratory output may be applied to a patient's cochlea via said supply electrode.

Additional aspects and corresponding advantages of the present invention will become apparent to those skilled in the art upon consideration of the further descriptions that follow.

### BRIEF DESCRIPTION OF THE DRAWINGS

FIG. 1 illustrates a fully implantable hearing instrument application comprising the present invention.

FIG. 2 illustrates one embodiment of a transducer and supportably interconnected, supply electrode employable in the application of FIG. 1.

FIG. 3 illustrates another embodiment of a transducer and supportably interconnected, supply electrode employable in the application of FIG. 1.

FIG. 4 is a side cross-sectional view of the embodiment of FIG. 3.

FIG. 5 illustrates yet another embodiment of a transducer and supportably interconnected, supply electrode employable in the application of FIG. 1.

FIG. 6 illustrates yet another embodiment of a transducer and supportably interconnected, supply electrode employable in the application of FIG. 1.

FIG. 7 illustrates a semi-implantable application comprising the present invention.

FIG. 8 illustrates a side cross-sectional view of one embodiment of an integrated transducer and supply electrode employable in the application of FIG. 7.

FIG. 9 illustrates a side cross-sectional view of another embodiment of an integrated transducer and supply electrode employable in the application of FIG. 7.

### DETAILED DESCRIPTION

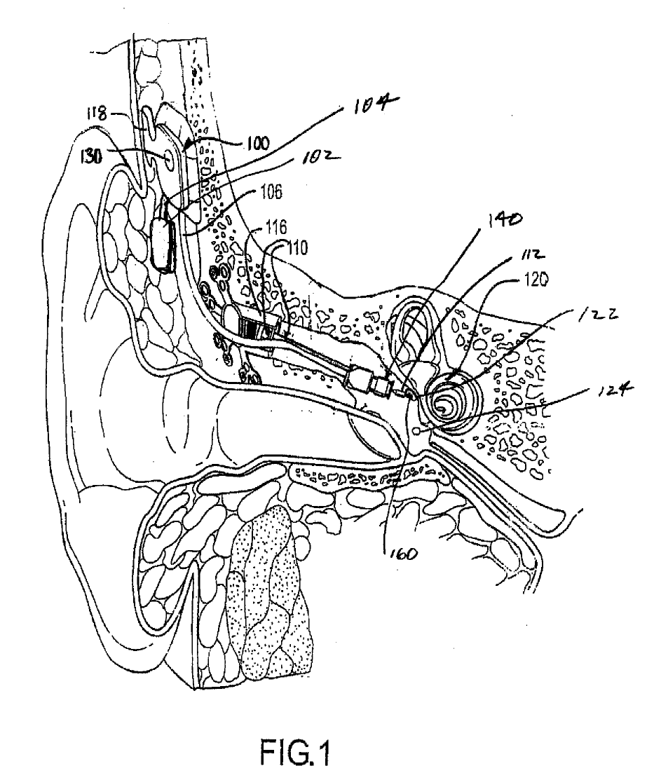

FIG. 1 illustrates one application of the present invention. As illustrated, the application comprises a fully implantable hearing instrument system. As will be appreciated, the present invention may also be employed in conjunction with semi-implantable hearing instruments. In the embodiment shown, the ossicular chain has been removed for purposes of illustration. It should be appreciated, however, that the embodiment(s) described herein may also be employed with all or portions of the ossicular chain present.

In the illustrated system, a biocompatible implant housing 100 is located subcutaneously on a patient's skull. The implant housing 100 may include a signal receiver 118 (e.g., comprising a coil element) and a pendant microphone 130 that is positioned to receive acoustic signals through overlying tissue. The signal receiver 118 may be utilized for transcutaneously re-charging an energy storage device within the implant housing 100 (e.g. via inductive coupling with an external charging device), as well as for receiving program instructions for the hearing instrument system.

The implant housing 100 may be utilized to house a number of components of the fully implantable hearing instrument. For instance, the implant housing 100 may house an energy storage device (e.g. a rechargeable battery), a microphone 130, and one or more signal processor(s). Various additional processing logic and/or circuitry components may also be included in the implant housing 100 as a matter of design choice. The signal processor(s) within the implant housing 100 may be electrically interconnected, e.g. via cable 106, to a biocompatible electromechanical transducer 140 and to an electrical supply electrode 160 that may be supportably interconnected to, defined by a portion of and/or in vibratory engagement with the electromechanical transducer 140, wherein the electromechanical transducer 140 may provide a vibratory output in response to a first electrical drive signal and the supply electrode 160 may provide an electrical output in response to a second electrical drive signal. In the later regard, the system may further include a return, or reference, electrode 102 positioned on the skull of a patient and electrically interconnected via electrical line 104 to circuitry within implant housing 100 used to generate the second drive signal.

The electromechanical transducer 140 may be supportably connected to a positioning system 110, which in turn may be connected to a bone anchor 116 mounted within the patient's mastoid process (e.g., via a hole drilled through the skull). The transducer 140 may include a vibratory member 112 for operatively interfacing the transducer 140 with a cochlea 120 of the patient. In an operative state, the vibratory member 112 provides a communication path for vibratory output from the transducer 140 and mechanical stimulation of the cochlea 120, e.g. through the transmission of vibrations to an oval window 122, round window 124, semicircular canal, bony exterior or an artificial fenestration of the patient's cochlea. As will be more fully discussed hereinbelow, the vibratory member 112 may also define, support and/or otherwise be in vibratory engagement with the supply electrode 160 for electrical stimulation of the cochlea 120.

During normal operation, acoustic signals are received subcutaneously at the microphone 130. Upon receipt of the acoustic signals, one or more signal processor(s) within the implant housing 100 processes the acoustic signals to provide processed electrical drive signals, e.g., a first drive signal to transducer 140 and a second drive signal to the supply electrode 160. As will be appreciated, the signal processor(s) may utilize digital processing techniques to provide frequency shaping, amplification, compression, and other signal conditioning, including conditioning based on patient-specific fitting parameters. The first drive signal causes the vibratory member 112 of the transducer 140 to output mechanical vibrations at acoustic frequencies to effect the desired sound sensation via mechanical stimulation of the cochlea 120 at the oval window 122 or round window 124 of the patient. Further, the second drive signal causes the supply electrode 160 to provide an electrical output at acoustic frequencies to electrically stimulate the cochlea 120 at oval window 122 or round window 124 of the patient.

As may be appreciated, the first and second drive signals may be separately provided to mechanically and electrically stimulate the cochlea 120 across corresponding first and second predetermined frequency ranges, respectively. For example, the first and second predetermined frequency ranges may be at least partially non-overlapping, wherein the first frequency range includes lower frequencies not included in the second frequency range, and wherein the second frequency range includes higher frequencies not included within the first frequency range. Further, the first and second frequency ranges may be established to be partially overlapping. In one implementation, the first frequency range may be established to extend from about 20 Hz to 2000 Hz, and the second frequency range may be established to extend from about 1000 Hz to 10,000 Hz.

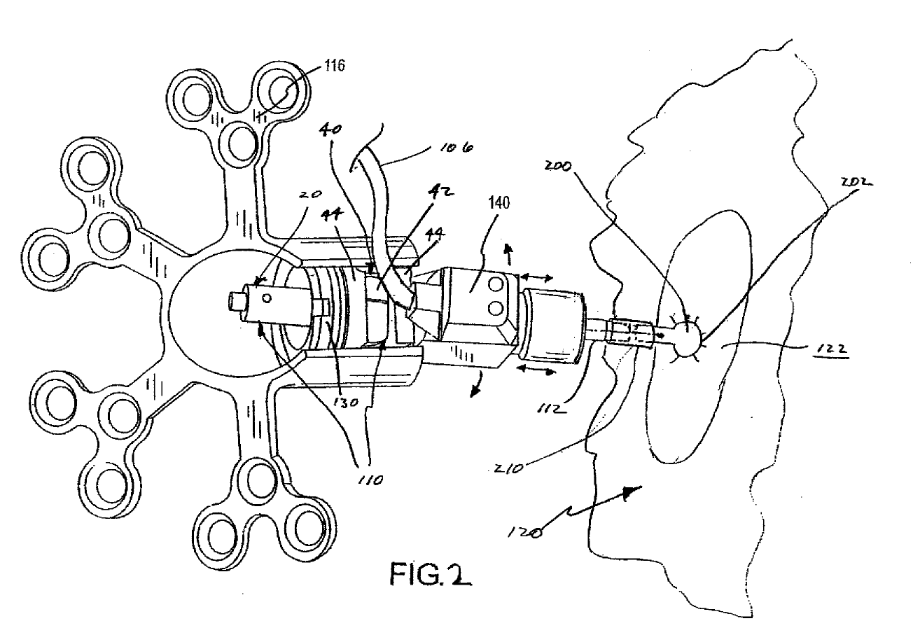

Reference is now made to FIG. 2, which illustrates an embodiment having an electrically conductive supply electrode 200 supportably interconnected to a vibratory member 112 of electromechanical transducer 140. More particularly, the supply electrode 200 may comprise an electrically conductive, biocompatible material (e.g. titanium) that defines a distal end which is fixedly interconnected via an electrically non-conductive, biocompatible, intermediate member 210 (e.g. comprising a ceramic material) to vibratory member 112. As illustrated via phantom lines in FIG. 2, the intermediate member 210 may comprise cup-shaped recesses at opposing ends to fixedly receive vibratory member 112 and supply electrode 200 therein.

As further illustrated, the supply electrode 200 may comprise an arcuate, or bulbous surface 202 for engaging a patient's cochlea 120, e.g. the oval window 122. Preferably, the supply electrode 200 may be positioned so that the arcuate surface 202 maintains contact with the patient's cochlea 120 during operations. For example, the supply electrode 200 may be implanted through a facial recess of the patient and brought into contact against the membrane of the round window 124. Fascia may be interposed between the supply electrode 200 and membrane. In another approach, the supply electrode 200 may be provided on an elongated wire that defines or is interconnected to the vibratory member 112. The supply electrode may be positioned and loaded against the oval window 122, or alternatively, against a bony exterior of the cochlea, the cochlear semicircular canal, a fenestration in the oval window 122 or a piston prosthesis that has been inserted through a fenestration in the oval window 124.

In this embodiment, cable 106 provides a first electrical drive signal to the electromechanical transducer 140 to affect vibrational output by vibratory member 112. Cable 106 further provides a second electrical drive signal to supply electrode 200 to yield an electrical output (e.g. as illustrated in FIG. 2 by a central phantom line that passes through vibratory member 112 and intermediate member 210 to contact supply electrode 200).

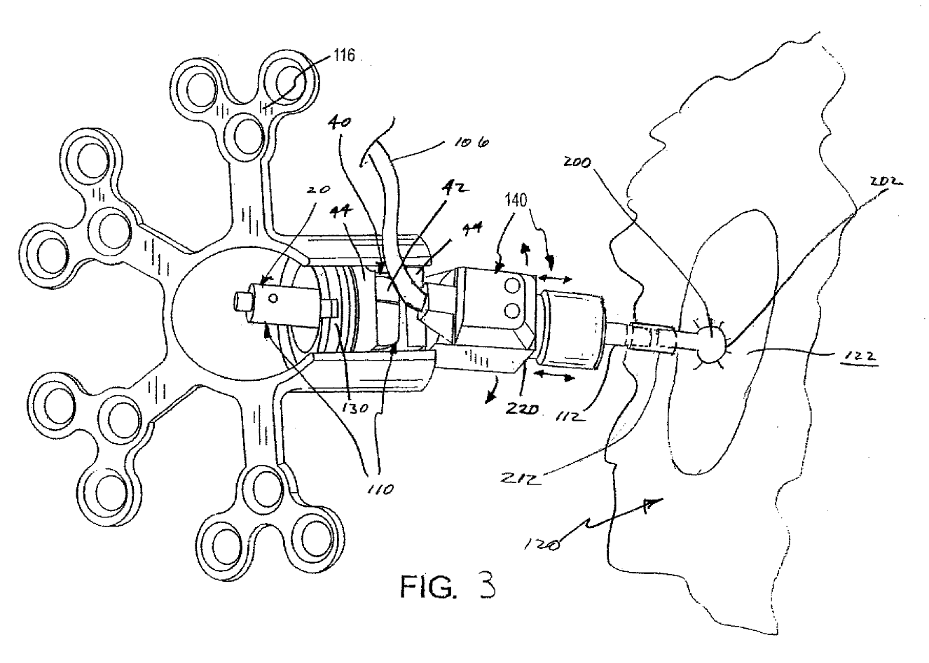

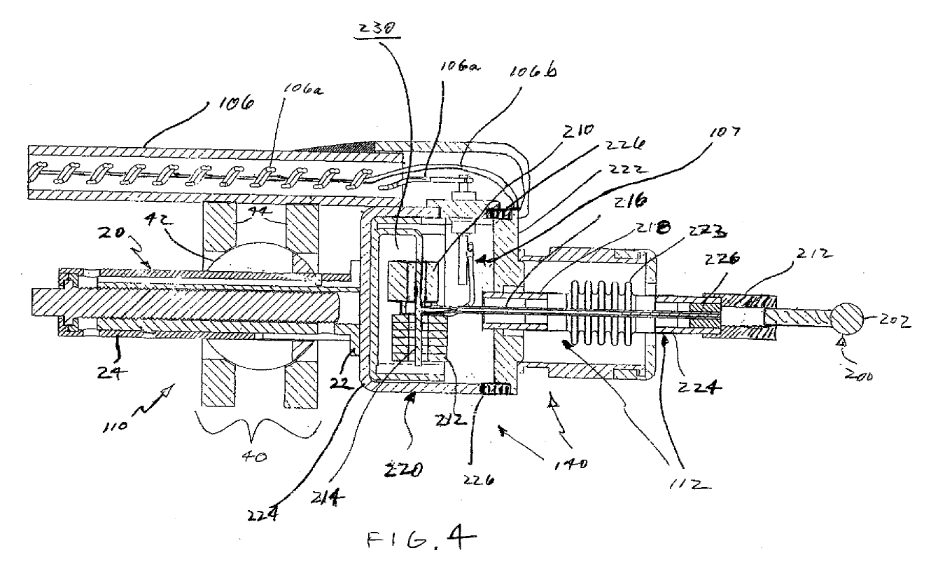

Reference is now made to FIGS. 3 and 4 which illustrate an embodiment similar to that illustrated in FIG. 2, wherein common componentry is referenced utilizing the same reference numerals as utilized in relation to the embodiment of FIG. 2. In contrast to the FIG. 2 embodiment, the embodiment of FIGS. 3 and 4 includes a supply electrode 200 that is fixedly interconnected to vibratory member 112 via an electrically conductive intermediate member 212. The vibratory member 112 is also electrically conductive, wherein the supply electrode 200 is electrically interconnected to an electrically conductive component of an external housing 220 comprising the electromechanically transducer 140. In this regard, and referring particularly to FIG. 4, the transducer housing 220 may comprise an electrically conductive end member 222.

Referring further to FIG. 4, in this embodiment the cable 106 includes a first electrical signal line 106a and a second electrical signal line 106b for conveying a first drive signal and second drive signal, respectively. The first signal line 106a is electrically interconnected via electrical lead 107 to an active element disposed within the housing 220, and the second signal line 106b is electrically interconnected to the housing end member 222 for conveying the second drive signal to the supply electrode 200 via the electrically conductive vibratory member 112 and intermediate member 212.

The end member 222 of housing 220 is interconnected to an electrically conductive cup member 224 with an electrically non-conductive member 226 interposed therebetween for isolation purposes. In turn, a hermetically sealed chamber 230 is defined within the housing 220. The housing 220 houses a magnetic coil 210, and stacked magnetic members 212, each of which extend about a leaf member 214, wherein the magnetic coil 210 and magnetic members 212 combinatively define active element that may be electrically driven to a generate a magnetic field to induce vibratory movement of the leaf member 214 at desired acoustic frequencies. In turn, the leaf member 214 may be interconnected to a drive pin 216, as shown.

Further in this regard, the drive pin 216 may be disposed to pass through an electrically conductive first plug member 218 of the vibratory member 112 that is proximally interconnected to the end member 222 of transducer housing 220 (e.g., via laser welding to yield a hermetic seal), an electrically conductive bellows member 223 of the vibratory member 112 that is proximally interconnected to a distal end of the first plug member 218 (e.g., via laser welding to yield a hermetic seal), and an electrically conductive second plug member 224 of the vibratory member 112 that is proximally interconnected to a distal end of the bellows member 223 (e.g., via laser welding to yield a hermetic seal.) The second plug member 224 may be distally interconnected to a distal end of the drive pin 216 via an electrically non-conductive, intermediate plug member 226 (e.g., a ceramic member interconnected to yield a hermetic seal), wherein the second plug member 224 may be axially displaceable with but is electrically isolated from the drive pin 216. In this regard the bellows member 223 may be provided with undulations that facilitate movement of drive pin 216 and the second plug member 224 relative to the transducer housing 220, while allowing the first plug member 218 to maintain a fixed position relative to the transducer housing 220. As shown, the intermediate member 212 may be interconnected to a distal end of the second plug member 224 and may include a slotted portion for receiving the supply electrode 200. More particularly, the supply electrode 200 may be inserted into the slotted portion of the intermediate member 212, wherein an outside surface of the slotted portion of the intermediate member 212 may be crimped to maintain the supply electrode 200 at desired fixed position relative to the intermediate member 212.

As noted above, the electromechanical transducer 140 may be supportably interconnected to a positioning system 110 that is supportably interconnected to a mounting member, or bone anchor 116. The bone anchor 116 may be of a type as taught in U.S. Pat. No. 6,293,903 entitled “APPARATUS AND METHOD FOR MOUNTING IMPLANTABLE HEARING AID DEVICE”, issued Sep. 25, 2001, the entirety of which is hereby incorporated by reference. Further, the positioning system 110 may be of the type as generally taught by U.S. Pat. No. 6,491,622 entitled “APPARATUS AND METHOD FOR POSITIONING AN IMPLANTABLE HEARING AID DEVICE” issued Dec. 10, 2002, the entirety of which is hereby incorporated by reference.

As best shown in FIG. 4, the positioning system 110 may include a carrier assembly 20 and a swivel assembly 40 that allow for selective three-dimensional positioning of the electromechanical transducer 140, and interconnected vibratory member 112 and supply electrode 200, at a desired location within a patient. In this regard, an external member 24 of the carrier assembly 20 may be supportively received and selectively secured in an opening defined through a split ball member 42 that is captured between plates 44 of the swivel assembly 40. The interface between the carrier assembly 20 and swivel assembly 40 provides for pivotable, lateral positioning of the transducer 140. That is, the carrier assembly 20 may pivot upon rotation of the ball member 42, thereby allowing the vibratory member 112 and supply electrode 200 to be moved along an arcuate path to a desired position. In turn, the interconnected plates 44 may be selectively secured to a bone anchor 116 and clamped via a lock member 130 (shown in FIG. 3) to compress the split ball member 42 and thereby maintain a selected pivotal orientation. At the same time, the carrier assembly 20 may be selectively secured along a continuum positions within the opening of the ball member 42, thereby facilitating linear positioning of the interconnected transducer 140, vibratory member 112 and supply electrode 200 in a depth dimension. Additionally, the carrier assembly 20 may be defined so that an internal member 22 thereof, connected to the transducer 140, may be selectively advanced and retracted in the depth of dimension relative to an external member 24 (e.g., by utilizing a lead screw arrangement), thereby further facilitating selective linear positioning of the transducer 140, vibratory member 112 and supply electrode 200.

As may be appreciated, in relation to an implementation shown in FIGS. 3 and 4, the positioning system 110 may be employed to move (e.g., advance or retract) the distal end of supply electrode 200 toward a patient's cochlea 120 by moving the carrier assembly 20 relative to the swivel assembly 40, by moving the internal member 22 of the carrier assembly 20 relative to the external member 24 thereof, and/or by pivoting the carrier assembly 20 relative to the swivel assembly 40 and mounting member 116.

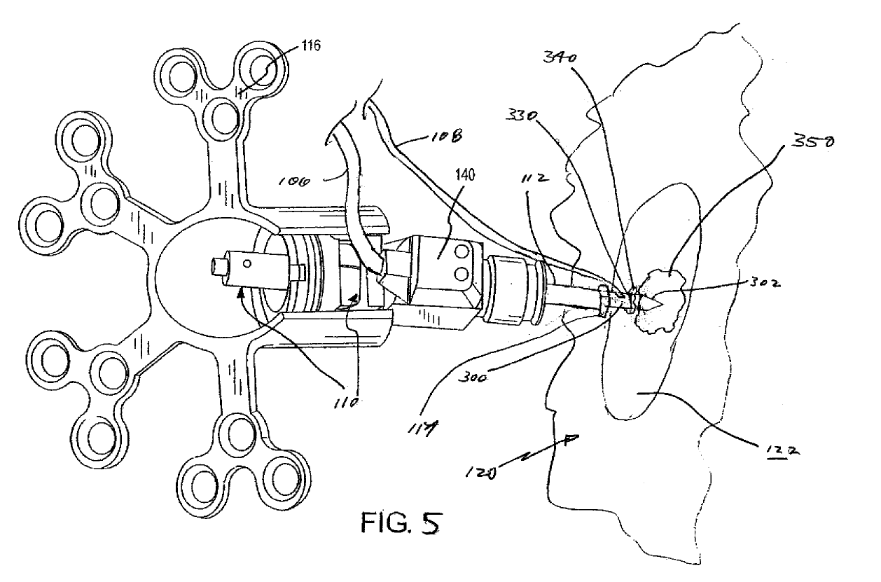

Reference is now made to FIG. 5, which illustrates another embodiment having a supply electrode 300 supportably interconnected to a vibratory member 112 of an electromechanical transducer 140. In this embodiment, the supply electrode 300 is partially inserted and thereby positioned within a patient's cochlea 120, e.g. through a small incision 330 in the membrane of the oval window 122 or round window 124, or in the semicircular canal, bony exterior or an artificial fenestration of the patient's cochlea. For purposes of illustration in FIG. 5, a portion 350 of the oval window 122 is cut-away to show the internal positioning of the supply electrode 300. The supply electrode 300 may comprise a surface 302 adapted to facilitate insertion into the oval window 122 of a patient. For example, the supply electrode 300 may comprise a tapered surface 302 that is advanced into the small incision 330 that is made through a patient's oval window 122 during implantation. As shown, fascia or other autologous tissue 340 has been introduced around the supply electrode 300 to facilitate sealing.

The supply electrode 300 may comprise an electrically conductive material that defines a distal end. The supply electrode 300 may be provided in the form of a separately positionable piston prosthesis with an end adapted to facilitate selective interconnection to a bail 114 provided at a distal end of the vibratory member 112.

In this embodiment, cable 106 provides a first electrical drive signal to the electromechanical transducer 140 to affect vibrational output by vibratory member 112. Additionally, a cable 108 may operatively interconnect the processor(s) of implant housing 100 to supply electrode 300 so as to provide a second electrical drive signal to supply electrode 300 to yield an electrical output.

As with the embodiments described in relation to FIGS. 1-4, transducer 140 may be supportably interconnected to a positioning system 110. The positioning system may be supportably interconnected to a mounting member or bone anchor 116 and may otherwise be provided to facilitate selective positioning of the transducer 140 and supportably interconnected Vibratory member 112 and supply electrode 300.

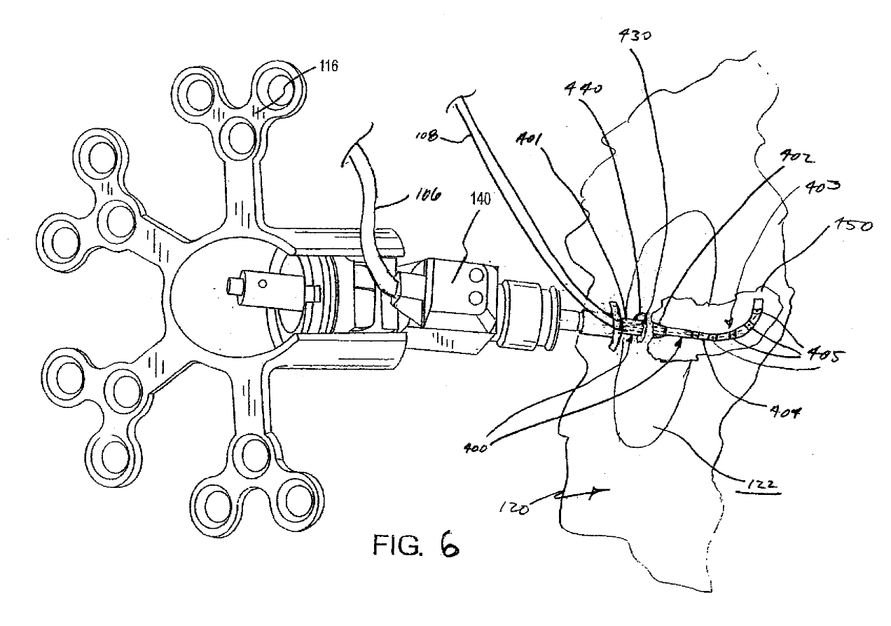

Reference is now made to FIG. 6, which illustrates another embodiment having a supply electrode 400 supportably interconnected to a vibratory member 112 of an electromechanical transducer 140. In this embodiment, supply electrode 400 is partially positioned within a patient's cochlea 120 through a small incision 430 in the oval window 122 thereof, and for purposes of illustration, a portion 450 of the oval window 122 is cut-away to show the internal positioning of the supply electrode 400. As shown, fascia or other autologous tissue 440 has been introduced around the supply electrode 400 to facilitate sealing.

The supply electrode 400 may comprise a proximal portion 401 and distal portion 403. The distal portion 403 may be flexible, jointed or otherwise curved to facilitate insertion into a curved portion of a patient's cochlea 120. The distal portion 403 may comprise a plurality of electrically-conductive electrode elements 405 disposed on a flexible member 404. By way of example, electrode elements 405 may comprise 12 pairs of bipolar electrodes and/or 8 to 22 monopolar electrodes with a reference electrode. For a short insertion electrode, at least one of a set of 6 bipolar and a set of 6 monopolar electrodes with a reference electrode may also be of significant benefit.

As illustrated, the distal portion 403 is supportably interconnected to and extends away from the proximal portion 401. In this regard, the proximal portion 401 may comprise an electrically non-conductive material. Further, the proximal portion 401 may comprise a distal end 402 having a reduced cross-section, e.g. a tapered surface 402 to facilitate insertion into the oval window 122 or round window 124 of a patient's cochlea 120. The proximal portion 401 may be selectively interconnected to a bail 114 provided at a distal tip of the vibratory member 112.

In this embodiment, cable 106 provides a first electrical drive signal to the electromechanical transducer 140 to affect vibrational output by vibratory member 112. Additionally, cable 108 may operatively interconnect the processor(s) of implant housing 100 to supply electrode 400 so as to provide a second electrical drive signal to supply electrode 400 to yield an electrical output.

As with the embodiments described in relations to FIGS. 1-5, transducer 140 may be supportably interconnected to a positioning system 110. The positioning system 110 may be supportably interconnected to a mounting member or bone anchor 116 and may otherwise be provided to facilitate selective positioning of the transducer 140 and supportably interconnected vibratory member 112 and supply electrode 400.

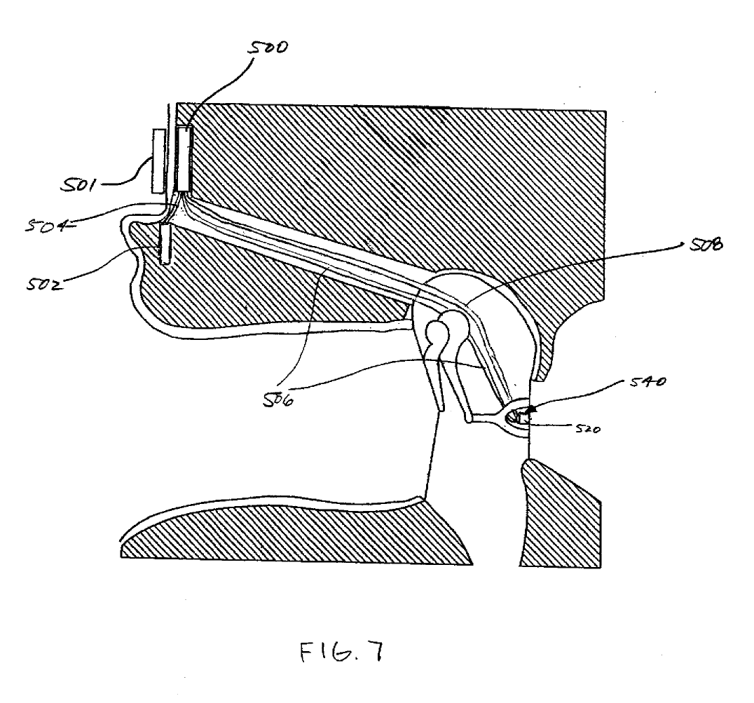

FIG. 7 illustrates another application of the present invention. As illustrated, this application comprises a partially implantable hearing instrument system. As previously noted, the present invention may be employed in conjunction with partially implantable or fully-implantable hearing instruments.

In the illustrated system, a bio-compatible implant housing 500 is located subcutaneously on a patient's skull. The implant housing 500 includes a signal receiver (e.g. comprising a coil element) for transcutaneous receipt of wireless signals (e.g. radio frequency signals) from an external unit 501. The external unit 501 may comprise a microphone for receiving acoustic signals, one or more signal processor(s) for processing electrical output signals from the microphone, and a coil for receiving processor signals and transcutaneous signal transmission to the implanted signal receiver (e.g. via inductive coupling). Additional external componentry may include a charging device for transcutaneously re-charging an implanted energy storage device (e.g. a rechargeable battery located within implant housing 100) via inductive coupling. The implant housing 500 may also include one or more signal processor(s) and associated circuitry that is electrically interconnected via cables 506, 508 to an integrated electrical and mechanical stimulation unit 540 attached to an oval window 122 of a patient. By way of example, the integrated unit 540 may be attached to the oval window 122 with an adhesive, glue, suture or the like. As will be further described, the integrated unit 540 is operable to provide a vibratory output and an electrical output directly to the cochlea of a patient.

In this regard, the integrated unit 540 may comprise a floating mass transducer for providing a vibratory output in response to a first electrical signal conveyed by cable 506, wherein the integrated unit 540 includes an external housing 520 that defines a hermetically-sealed internal chamber therewithin, and an active transducer element located within the internal chamber for receiving the first electrical signal. Further, in the illustrated embodiment the external housing 520 may be electrically conductive to integrally define a supply electrode for providing an electrical output in response to a second electrical signal conveyed by cable 508. In this regard, the system may further include a return or reference electrode 502 positionable on the skull of a patient and electrically interconnected via electrical line 504 to circuitry within implant housing 500 used to generate the second drive signal.

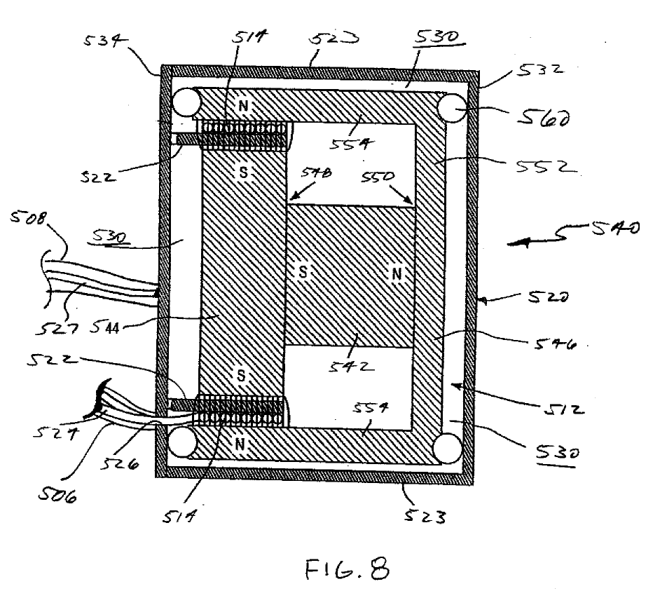

Reference is now made to FIG. 8 which illustrates an embodiment of an integrated unit 540 having an integrated floating mass transducer and supply electrode. In this embodiment, the integrated unit 500 comprises a sealed, electrically conductive housing 520 that integrally defines a supply electrode and that houses a magnet assembly 512 and a coil 514. The magnet assembly 512 may be loosely suspended within the housing 520, and the coil 514 may be rigidly secured to the housing 520. The magnet assembly 512 may include a permanent magnet 542 and associated pole pieces 544 and 546. When a first electrical drive signal (e.g. an alternating current) is conducted to the coil 512 via electrical line 524 of cable 506, the coil 512 and magnet assembly 514 oscillate relative to each other and cause the housing 520 to vibrate. In this regard, the coil 512 acts as the active element and magnet assembly 514 acts as the floating mass component of the transducer.

The exemplary housing 520 may be a cylindrical capsule having a diameter of 1 mm and a thickness of 1 mm, and may be made from an electrically conductive, biocompatible material such as titanium. The housing 520 may define first and second faces 532, 534 that are substantially parallel to one another, and an outer wall 523 which is substantially perpendicular to the faces 532, 534. An electrically non-conductive interior wall 522 may be affixed to the interior of the housing 520 and may define a circular region which runs substantially parallel to the outer wall 523.

The housing 520 may define a sealed chamber 530 having air spaces that surrounds the magnet assembly 512 so as to separate it from the interior of the housing 520 and allow it to oscillate freely without colliding with the coil 514 or housing 520. The magnet assembly 512 may connected to the interior of the housing 520 by flexible membranes such as silicone buttons 560. The magnet assembly 512 may alternatively be floated on a gelatinous medium such as silicon gel which fills the air spaces in the housing 520. A substantially uniform flux field may be produced by configuring the magnet assembly 512 as shown in FIG. 8. In this regard, the assembly 512 may include a permanent magnet 542 positioned with ends 548, 550 containing the south and north poles substantially parallel to the circular faces 534, 532 of the housing 520. A first cylindrical pole piece 544 may be connected to the end 548 containing the south pole of the magnet 542 and a second pole piece 546 may be connected to the end 550 containing the north pole. The first pole piece 544 may be oriented with its circular faces substantially parallel to the circular faces 532, 534 of the housing 520. The second pole piece 546 has a circular face which has a rectangular cross-section and which may be located substantially parallel to the circular faces 532, 534 of the housing 520. The second pole piece 546 may also comprise a pair of walls 554 which are parallel to the wall 523 of the housing 520 and which surrounds the first pole piece 544 and the permanent magnet 542.

The coil 514 partially encircles the magnet assembly 512 and is fixed to the interior wall 522 of the housing 510 such that the coil 514 is more rigidly fixed to the housing 520 than the magnet assembly 512. In one implementation, a pair of leads 524 of cable 506 are connected to the coil 514 and pass through an opening 526 in the housing 520 to the exterior of the housing 520. The cable 506, a first electrical drive signal, e.g. delivers an alternating current signal to the coil 514 via the leads 524. The opening 526 is closed around the leads 524 to form a seal (not shown) which prevents contaminants from entering the housing 520. As shown in FIG. 8, cable 508 may be physically interconnected to the electrically conductive housing 520, wherein an electrical line 522 of cable 508 may convey a second electrical drive signal to the housing 520. In turn, the housing 520 functions as the supply electrode to provide an electrical output for electrical stimulation of a patient's cochlea.

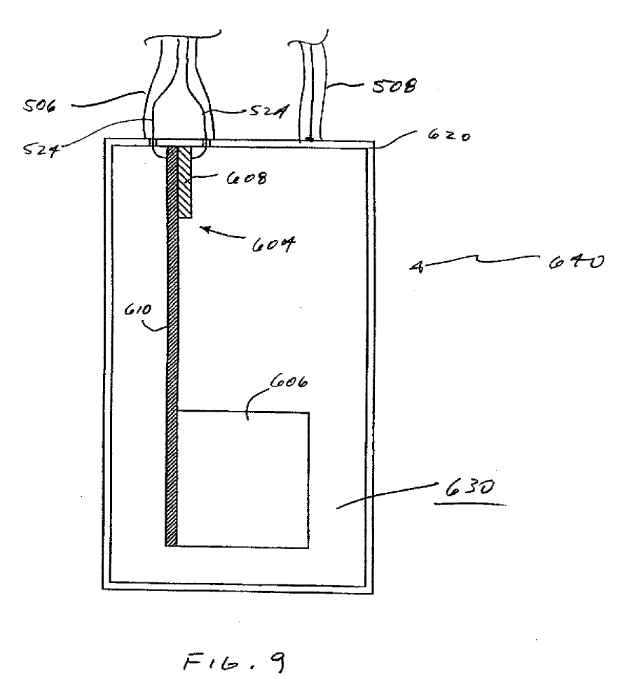

Reference is now made to FIG. 9 which illustrates another embodiment of an integrated unit 640 having an integrated transducer and supply electrode. In this embodiment, a floating mass is caused to vibrate by a piezoelectric bimorph. More particularly, the integrated unit 640 comprises an electrically conductive housing 620 that integrally defines a supply electrode and that houses a bimorph assembly 604 and a driving weight 606 within an internal chamber 630. One end of the bimorph assembly 604 may be secured to the inside of the housing 620 and may comprise a short piezoelectric strip 608 and a longer piezoelectric strip 610. The two strips are oriented so that one strip contracts while the other expands when a voltage is applied across the strips via electrical leads 524 of cable 506.

A driving weight 606 may be secured to one end of piezoelectric strip 610 (the “cantilever”). When a first drive signal (e.g. an alternating current) is conducted to the bimorph assembly 604 via leads 524, the housing 620 and driving weight 600 oscillate relative to each other causing the housing 620 to vibrate. Preferably, the relative vibration of the housing 620 is substantially greater than the vibration of the driving weight 606.

As shown in FIG. 9, cable 508 may be physically interconnected to the electrically conductive housing 620, wherein an electrical line 527 of cable 508 may convey a second electrical drive signal to the housing 620. In turn, the housing 620 functions as the supply electrode to provide an electrical output for electrical stimulation of a patient's cochlea.

The descriptions of the various embodiments hereinabove are for purposes of illustration and are not intended to limit the scope of the present invention. Various adaptations and modifications are intended to be with the scope of the present invention as defined by the claims which follow.

1. An implantable hearing instrument comprising:
an electromechanical transducer for providing a vibratory output in response to a first electrical signal, corresponding with an acoustic signal, for mechanical stimulation of a patient's cochlea; and,
a supply electrode, supportably interconnected to said electromechanical transducer, for electrically stimulating a patient's cochlea in response to a second electrical signal corresponding with said acoustic signal.

2. An implantable hearing instrument as recited in claim 1, wherein said supply electrode is interconnected to said transducer for movement responsive to said vibratory output.

3. An implantable hearing instrument as recited in claim 2, wherein said transducer comprises:
a housing; and,
a vibratory member, supportably interconnected to said housing for movement relative thereto in response to said vibratory output, wherein said supply electrode is supportably interconnected to said vibratory member.

4. An implantable hearing instrument as recited in claim 3, wherein said supply electrode defines a distal end for engaging a patient's cochlea.

5. An implantable hearing instrument as recited in claim 4, wherein said distal end comprises an arcuate surface for engaging an outer surface of a patient's cochlea.

6. An implantable hearing instrument as recited in claim 4, wherein said distal end is adapted for insertion into a patient's cochlea.

7. An implantable hearing instrument as recited in claim 3, wherein said supply electrode comprises a distal portion and a proximal portion that is supportably interconnected to said vibratory member.

8. An implantable hearing instrument as recited in claim 7, wherein said distal portion of said supply electrode is at least one of flexible, jointed and curved for positioning within a curved portion of a patient's cochlea.

9. An implantable hearing instrument as recited in claim 8, wherein a distal portion of said supply electrode comprises:
a plurality of electrode elements spaced along said distal portion.

10. An implantable hearing instrument as recited in claim 2, wherein said transducer comprises:
a housing; and,
a vibratory member, supportably interconnected to said housing for movement relative thereto in response to said vibratory output, wherein said vibratory member is electrically-conductive and integrally defines said supply electrode.

11. An implantable hearing instrument as recited in claim 1, wherein said supply electrode is adapted to electrically stimulate said patient's cochlea across a first frequency range in response to said first electrical signal, wherein said electromechanical transducer is adapted to vibrate said vibratory member to mechanically stimulate said patient's cochlea across a second frequency range in response to said second electrical signal, and wherein said first and second frequency ranges are at least partially non-overlapping.

12. An implantable hearing instrument as recited in claim 10, wherein said first and second frequency ranges are at least partially overlapping.

13. An implantable hearing instrument as recited in claim 1, further comprising:
a mounting member interconnectable in fixed relation to a patient's skull and adapted for supportable interconnection of said electromechanical transducer thereto.

14. An implantable hearing instrument as recited in claim 13, wherein a positioning assembly is interconnectable to said mounting member and adapts to supportably and selectively position, said electromechanical transducer relative to a patient's cochlea.

15. An implantable hearing instrument as recited in claim 13, wherein said transducer comprises:
a housing; and,
a vibratory member, supportably interconnected to said housing for movement relative thereto in response to said vibratory output wherein said supply electrode is supportably interconnected to said vibratory member.

16. An implantable hearing instrument as recited in claim 15, wherein said supply electrode is supportably interconnected at a distal end of said vibratory member.

17. An implantable hearing instrument as recited in claim 13, further comprising:
an implantable stimulation source for contemporaneously supply said first and second electrical signals in response to electrical audio signals corresponding with external acoustic signals.

18. An implantable hearing instrument as recited in claim 13, wherein said stimulation source processes said audio signals utilizing stored algorithms.

19. An implantable hearing instrument as recited in claim 18, further comprising:
an implantable microphone for receiving acoustic signals and providing said audio signals to said signal source for use in supplying said first and second electrical signal.

20.-64. (canceled)
